SEQUENCE LISTING (1 sequence)

SEQ ID NO: 1 - protein, 30 aa, Homo sapiens
FIHCPTENEPDLAQCFFCFKELEGWEPDDD

(19) United States
(12) Patent Application Publication
(10) Pub. No.: US 2017/0296639 A1
(43) Pub. Date: Oct. 19, 2017

(54) METHOD FOR PREPARING DENDRITIC CELL LOADED WITH ANTIGEN

(71) Applicants: Shenzhen Institutes of Advanced Technology, Shenzhen, Guangdong (CN); Hryz Biotech Co., Shanghai (CN)

(72) Inventors: Yifan MA, Shenzhen (CN); Xiangjun ZHOU, Shenzhen (CN); Shang CHEN, Shenzhen (CN); Lintao CAI, Shenzhen (CN); Ce WANG, Shenzhen (CN); Peng LIU, Shenzhen (CN)

(21) Appl. No.: 15/312,976

(22) PCT Filed: May 20, 2015
(86) PCT No.: PCT/CN2015/079416

§ 371 Date: Nov. 21, 2016

(30) Foreign Application Priority Data
May 21, 2014 (CN) 201410216790.7

(51) Int. Cl. A61K 39/00; C12N 7/00; A61K 39/29; C12N 5/0784

(52) U.S. Cl. CPC A61K 39/0011; A61K 39/292; C12N 5/0639; C12N 7/00; C12N 2501/05; A61K 2039/5154; C12N 2501/22; C12N 2501/2304; C12N 2730/10134; C12N 2500/90

(57) ABSTRACT

The present invention provides a method for preparing dendritic cell loaded with antigen, the method comprising the steps of adding serum-free cell culture medium containing granulocyte-macrophage colony-stimulating factor (GM-CSF) and inter-leukin (IL)-4 into mononuclear cells, culturing in an incubator at 37° C. under 5% carbon dioxide for 5 days, adding target antigen wrapped cationic liposome and culturing for 8-24 hours to obtain target antigen loaded dendritic cell.

### TECHNICAL FIELD

The present invention relates to the field of cell immunotherapy, and particularly, to a method for preparing dendritic cells using cationic liposomes as antigen carrier.

### BACKGROUND

In recent years, the immune cell treatment is a research hotspot, attracting an increasingly attention in clinical applications. Since this treatment has no tremendous toxic and side effects caused by chemoradiotherapy, achieves relative safety and improves the life quality of patients, the treatment with dignity has been recognized as the most promising method of cancer treatment. Cancer immunotherapy represented with dendritic cell (DC) vaccines becomes the fourth anti-tumor therapy following the surgery, chemotherapy, and radiotherapy, which mainly uses the antigenic substance of tumor cells to stimulate the body to generate specific immunity destruction of tumor cells, so as to achieve the purpose of elimination of tumor. Compared with the traditional anti-tumor therapy, DC vaccine-mediated immunotherapy has many advantages such as good safety and high specificity, and now has been widely used in treatment for various malignant tumors such as lung cancer, colon cancer, prostate cancer, breast cancer, and melanoma, but its clinical efficacy needs to be further improved. As the most potent antigen-presenting cells in body, dendritic cell (DC) is the primary core of stimulating the body to generate anti-tumor immune response. It can take antigens, and present the antigenic information to CD8+T lymphocytes via MHC-I molecules, and it is capable of inducing generation of specific cytotoxic T lymphocytes (CTL), and then kill tumor cells, and is applicable in immunotherapy for various tumor. Therefore, increasing the uptake amount and presentation capabilities of DC to antigen is an important strategy to enhance their clinical efficacy.

Liposomal nanoparticle is spherical entity which is formed by the phospholipid bilayer shell encapsulating a water phase core, the structure of which is similar to a biomembrane, and is a biocompatible and non-toxic nanomaterial. It can encapsulate water-soluble and lipo-soluble drugs, with advantages such as reducing drug dose, sustained release, and targeted drug release, and therefore is widely used in development of nano anti-tumor drugs. In addition, nanoliposomes is also an excellent antigen carrier, not only encapsulating a series of antigens with different physicochemical properties and immune adjuvants, protecting protein polypeptide antigen from degradation, but also promoting phagocytosis and presentation of antigen-presenting cells to antigen, enhancing the specific immune response of the body. Based on these advantages, as a novel vaccine vector, liposomal nanoparticles are increasingly used for research and development of bacterial vaccines, viral vaccines, anti-parasite vaccines and anti-tumor vaccines and the like. Neutral liposomes and cationic liposomes whose surface carries positive charges are the most commonly used nano vaccine vector, wherein, the cationic liposomes are particularly worthy of attention, which is not only a excellent protein/peptide antigen carrier, but also a novel immunological adjuvant that may directly activate antigen presenting cells, and enhance vaccine-induced immune response. Currently, the cationic liposomes have been used for a new generation of influenza vaccine by GSK Company.

A preparation method of dendritic cell vaccine loaded with autologous tumor associated holoantigen (Publication No. CN102091327A), its solution is to collect mononuclear cells separated from human peripheral blood, induce the cultured DC cells, impact the DC cells using the prepared autologous tumor associated holoantigen, mature the DC cells and prepare autologous tumor antigen specific DC vaccine.

A preparation process of dendritic cell targeted nanoliposome tumor vaccine (Publication No. CN101690805A), its solution is to embed α1, 3 Gal sugar chain—triglycerides directly into the phospholipid bilayer of liposomes, prepare a nano-vaccine delivery system capable of targeting DCs; preparing nano tumor vaccine liposomes with DCs targeting effect by use of this vaccine delivery system loading tumor antigen to enhance uptake and presentation of DCs to tumor antigen, while Toll like receptor signaling pathway of DCs is actived and its maturation is promoted, and immune response of specific anti-tumor cells is further induced.

Dendritic cell tumor vaccine and preparation method thereof (Publication No. CN102793912A), the antigen composition loaded in the DC-tumor vaccine provided by this solution is derived from tumor tissues itself, mainly targeting at cancer stem cells, which is loaded with a dendritic cell tumor vaccine of a multi-medicine resistance tumor stem cell antigen composition.

A preparation method for dendritic cell of umbilical cord blood source and dendritic cell vaccine (Publication No. CN102676455A), its solution using stem cell growth factor and cytokine Flt3-L can effectively promote a hematopoietic cell in the umbilical cord blood to induce and proliferate to an immune cell to obtain DC, and then DC is stimulated with a tumor-specific antigen to obtain human dendritic cell tumor vaccine.

A preparation method for dendritic cell vaccine (Publication No. CN102847145A), its solution is recycling separated autologous plasma, replacing human plasma with in vitro human AB serum, adding Flt3 into the DC media, enhancing stimulation of dendritic cell expansion, using its own specific cancer stem cell lysates as a tumor antigen load to prepare autologous tumor vaccine.

A preparation method of high-activity antigen-loaded dendritic cell (Publication No. CN103013915A), its solution is collecting and separating mononuclear cells from peripheral blood to induce to obtain dendritic cells, adding the corresponding tumor antigen and M. tuberculosis purified protein derivative (PPD) to further culture, and then a high-activity antigen-loaded dendritic cell can be obtained.

None of the above solutions uses liposome with targeting effect as antigen carrier to load DC cells with antigen.

### SUMMARY

The object of the present invention is to provide a method of preparing dendritic cells by use of cationic liposome as antigen carrier, which is simple and available, with immune target diversity, strong antigenicity and good stability, and ease of operation and clinical applications.

To achieve the above object, the present invention provides a preparation method of dendritic cells loaded with antigens efficiently, characterized in that: the steps are adding serum-free cell culture medium containing GM-CSF and IL-4 into mononuclear cells, and placing in an incubator of 37° C., with 5% of CO₂ for culture; after 5 days, adding cationic liposomes that encapsulate target antigen to culture for 8-24 hours, and then dendritic cells loaded with the target antigen can be obtained.

The cationic liposome is a mannose-modified cationic liposome complex.

The cationic liposome is obtained by coupling a mannose or a mannoside to a polyethylene glycol derivatized phospholipid to obtain a mannose-modified polyethylene glycol derivatized phospholipid; dissolving a cationic lipid and the mannose-modified polyethylene glycol derivatized phospholipid into a mixed solvent of chloroform and methanol respectively, after mixing to obtain a mixture liquid; rotarily evaporating the mixture liquid with a steady nitrogen stream or an inert gas stream so as to form a uniform film; adding a PBS buffer solution containing tumor antigen after vacuum drying and placing at 4° C. for sonicating to hydrate; obtaining the cationic liposome after extruding through film; wherein the loading amount of tumor antigen is 1-500g antigen/mol liposome.

The range of mole ratio of the cationic lipid to the mannose-modified polyethylene glycol derivatized phospholipid is 1:1 to 1:10.

The cationic lipid is any one of didecyldimethylammonium bromide, dioleoyltrimethylammoniumpropane, dioleoylpropyltrimethylammonium chloride, 3-(N-(N′, N′-dimethylaminoethane) carbamoyl) cholesterol and dioleyl ether phosphatidylcholine.

The target antigen is one or more tumor antigen protein or polypeptide having different epitopes.

The antigen may be selected from the group consisting of tumor cell lysate, autologous or allogeneic tumor antigen protein, genetically engineered polypeptide or protein product and synthetic antigen polypeptide.

The antigen is HBsAg antigen, tumor tissue antigens, electroneutral polypeptide antigen, electronegative polypeptide antigen survivin or OVA protein antigen.

The DC cells include peripheral blood mononuclear cell induced DC cells, hematopoietic stem cell and umbilical cord blood stem cell induced DC cells.

Human peripheral blood mononuclear cells are suspended in basal medium, and then seeded into cell culture plates for attachment culture for 1-2 hours at 37° C. in incubator; after non-attached cells are removed, a serum-free cell culture medium containing GM-CSF and IL-4 is added into the attached cells to culture at 37° C., with 5% of CO₂, under saturated humidity for carrying out the induction of DC cells; on the third day, DC cell culture medium is supplemented to DC cell culture plate in half amount; on the fifth day, liposome-encapsulated antigen is added to DC cells, cultured for 8-24 hours, wherein the adjustment dose of antigen is 1-50 ug/ml, and DC cells loaded with tumor antigens are obtained.

Preferably, serum-free cell culture medium contains 25-500 ng/m1 of GM-CSF and 5-100 ng/ml of IL-4.

Currently, the present invention proposes the use of mannose-modified liposomes as antigen carrier, in order to improve the antigen loading efficiency and anti-tumor effect of DC cells, in connection with the problems such as the difficulty in loading antigen and low immune efficiency in the process of preparation of DC cells.

The cationic liposome of the present invention is characterized in that: it is a mannose-modified cationic liposome complex, the main component of which is cationic lipid and mannose-modified polyethylene glycol derivatized phospholipids. The cationic lipid is amphiphatic molecule, the surface of which carries positive charge. The main chain of molecule is glycerol, and the second hydroxyl group of the glycerol is quaternary ammonium salt with positive charge and the other two hydroxyl groups are esterified with saturated or unsaturated fatty acid. The cationic lipid having the above characteristics mainly includes: cationic lipid comprising didecyldimethylammonium bromide (simply referred as DDAB), dioleoyltrimethylammoniumpropane (simply referred as DOTAP), dioleoylpropyltrimethylammonium (simply referred as DOTMA), 3-(N-(N′, N′-dimethylaminoethane) carbamoyl) cholesterol (simply referred as DC-Chol) and dioleyl ether phosphatidylcholine (simply referred DOEPC).

Wherein, the range of mole ratio of the cationic lipid to mannose-modified polyethylene glycol derivatized phospholipid is 1:1 to 1:10.

The mannose comprises D-mannose, and other monosaccharides that can be identified by mannose receptor, such as D-galactose, 4-nitrophenyl-α-D-mannopyranoside, 4-am inophenyl α-D-mannopyranoside, 4-am inophenyl-β-D-galactopyranoside (Sigma-Aldrich), α-D-mannosylphenyl isothiocyanate, α-D-Galactopyranosylphenyl isothiocyanate.

The mannose-modified polyethylene glycol derivatized phospholipid is obtained by covalently coupling the mannose described in 5) to the DSPE-PEG molecules

The mannose-modified cationic liposome is characterized in that: it can simultaneously encapsulate one or more antigens, and can improve the antigen-loading efficiency and antigen-presenting capacity of human DC and enhance the anti-tumor effect thereof.

The cationic liposomes of the present invention have been disclosed in CN102973506A (Mar. 20, 2013 published).

Antigen loaded in the liposome is one or more tumor antigen proteins or polypeptides having different epitopes, and the antigen may be derived from tumor cell lysate, autologous or allogeneic tumor antigen protein, genetically engineered polypeptide or protein product, synthetic antigenic polypeptides and so on.

The human DC cells include peripheral blood mononuclear cells induced DC, hematopoietic stem cell and umbilical cord blood stem cell induced DC.

The present invention discloses a method of DC cells loaded with tumor antigen, using mannose-modified cationic liposome as antigen carrier to encapsulate tumor antigen for the preparation of DC cells. The cationic liposome comprises phospholipid bilayer ball, polyethylene glycol derivatized phospholipids and mannose. The phospholipid bilayer ball is composed of cationic lipids and phospholipid molecules in polyethylene glycol derivatized phospholipids. The mannose forms mannose-modified polyethylene glycol derivatized phospholipids at one end of the polyethylene glycol derivatized phospholipids. Such cationic liposomes are obtained by coupling mannose-modified polyethylene glycol derivatized phospholipids on the polar groups of phospholipid molecules. The tumor antigen is one or more tumor associated antigen protein or polypeptide having different epitopes. The antigen carrier technology combined with new nano liposome enhances the antigen load and activation of DC cell and improves the antigen-presenting efficiency of DC cell.

In the tumor environment, DC cell must take antigens and present it to CD8 and CD4T cells by MHC-I or-II molecules respectively, so as to trigger the body to produce anti-tumor immunity. However, the efficacy of tumor immunotherapy using DC is closely associated with the amount of DC loaded with tumor antigens, the quantity of DC activation and DC taking tumor antigens and presenting it to T-cell. Currently, the conventional preparation process for DC is firstly separating mononuclear cells from peripheral blood, and then inducing the mononuclear cells into DC cell with cytokine such as IL-4, GM-CSF and so on, followed by addition of tumor antigens obtained from lysis of tumor cells of patients so that DC is loaded with tumor antigens. Although this process can make DC take the tumor antigen of patient, due to weakness of antigenicity of tumor antigen, and singleness of target that activates immune, the uptake amount of DC to tumor antigens is less, and the ability to present antigen is also lower, so that killing activity of CTL cell generated from DC is insufficient, thereby affecting the effect of anti-tumor therapy.

Currently, the present invention proposes the use of mannose-modified liposomes as antigen carrier to encapsulate one or more tumor antigens, so that the anti-tumor effect of DCs is enhanced by improving the antigen loading efficiency and the antigen presentation thereof, in connection with problems such as the anti-tumor efficacy of existing DC cell being poor. The mannose-modified cationic liposome can not only load various antigens with different physical and chemical properties, but also has the ability to target DC, so as to greatly improve the uptake efficiency of DCs cell to tumor antigen and the ability of antigen presenting, so that anti-tumor immunization of DC cell is enhanced.

The present invention discloses a method of DCs loaded with tumor antigens, using mannose-modified cationic liposome as antigen carrier to encapsulate tumor antigen for loading DC cells with antigen. The cationic liposome comprises phospholipid bilayer ball, polyethylene glycol derivatized phospholipids and mannose; the phospholipid bilayer ball is composed of cationic lipids and phospholipid molecules in polyethylene glycol derivatized phospholipids; the mannose forms mannose-modified polyethylene glycol derivatized phospholipids at one end of the polyethylene glycol derivatized phospholipids. Such cationic liposomes are obtained by coupling with mannose-modified polyethylene glycol derivatized phospholipids at the polar groups of phospholipid molecules. The tumor antigen is one or more tumor antigen proteins or polypeptides having different epitopes.

A preparation method of mannose-modified cationic liposome, comprising the steps of:

(1) providing a polyethylene glycol derivatized phospholipid, coupling a mannose or a mannoside to the polyethylene glycol derivatized phospholipid, obtaining a mannose or mannose oligosaccharide-modified polyethylene glycol derivatized phospholipid;

(2) dissolving the cationic lipid and the mannose or mannoside modified polyethylene glycol derivatized phospholipid into a mixed solvent of chloroform and methanol respectively, after mixing to obtain a mixture liquid;

(3) vortically drying the mixture liquid with a steady nitrogen stream or an inert gas stream so as to form a uniform film; adding PBS buffer solution containing tumor antigen after vacuum drying and placing at 4° C. for ultrasound hydration; obtaining the cationic liposome after extruding through film.

A method of loading DC with antigen comprises the steps of:

(1) suspending a human peripheral blood mononuclear cells in basal medium, and then inoculating the human peripheral blood mononuclear cells into cell culture plates for attachment culture for 1-2 hours at 37° C. in incubator.

(2) removing the non-attached cells and adding DC cell culture medium into the attached cells, to culture at 37° C., with 5% of CO₂, under saturated humidity for carrying out the induction of DC cell. On the third day, DC cell culture medium is supplemented to DC cell culture plate in half amount. On the fifth day, suitable amount of liposome-encapsulated antigen is added to DC cells, cultured for 8-24 hours, and DC cells loaded with tumor antigen are obtained.

The antigen carrier technology using new nano liposome can enhance the antigen load and antigen-presenting of DC cell, thereby enhancing the anti-tumor efficacy of DC vaccines.

In order to solve the above problems in the prior art, the technical solution provided by the present invention is:

using mannose-modified cationic liposome as antigen carrier to encapsulate tumor antigen is used for preparation of DC cell. The cationic liposome comprises phospholipid bilayer ball, polyethylene glycol derivatized phospholipids and mannose; the phospholipid bilayer ball is composed of cationic lipids and phospholipid molecules in polyethylene glycol derivatized phospholipids; the mannose forms mannose-modified polyethylene glycol derivatized phospholipids at one end of the polyethylene glycol derivatized phospholipids. Such cationic liposomes are coupled with mannose-modified polyethylene glycol derivatized phospholipids at the polar groups of phospholipid molecules. The tumor antigen is one or more tumor associated and specific antigen protein or polypeptide having different epitopes.

A cationic liposome which encapsulates antigen, in one embodiment shown in FIG. 1, comprises phospholipid bilayer ball, polyethylene glycol derivatized phospholipids and mannose. The phospholipid bilayer ball is composed of cationic lipids and phospholipid molecules in polyethylene glycol derivatized phospholipids. The mannose is coupled to one end of the polyethylene glycol derivatized phospholipids to form mannose-modified polyethylene glycol derivatized phospholipids. In this embodiment, the inner side of the phospholipid bilayer ball is coated with tumor antigen, wherein the antigens may be one or more combination of proteins or polypeptides. Antigens can be separated and extracted directly from tumor, viruses, bacteria, or other microorganisms, or may be protein/polypeptide product that can be obtained by genetic engineering, or may be synthesized polypeptide.

Polyethylene glycol derivatized phospholipid is obtained by coupling a phospholipid to one end of polyethylene glycol (PEG) and amination of the other end. One end of polyethylene glycol in the present embodiment is coupled with distearoylphosphatidylethanolamine (DSPE), referred as DSPE-PEG. The molecular weight of polyethylene glycol in polyethylene glycol derivatized phospholipid is preferably from 1,000 to 5,000. The ratio range of molar number of polyethylene glycol derivatized phospholipid to cationic lipid is 1:5 to 1:50. Mannose is coupled to the amination end of polyethylene glycol derivatized phospholipids in covalent manner. The cationic lipid is amphiphatic molecule, the surface of which carries positive charge. The main chain of the cationic lipid is glycerol, and the center hydroxyl group in the three hydroxyl groups of the glycerol is connected with quaternary ammonium salt with positive charge and the other two hydroxyl groups are esterified with saturated or unsaturated fatty acid.

Liposome nano vaccine vector with targeting effect is characterized in that the specific operation of the method is as follows.

Mannose modified PEG-Mannose is first prepared. A series of monosaccharide that can be recognized by mannose receptor such as mannose is attached to the amino group of DSPE-PEG-NH₂ in covalent manner, so as to obtain DSPE-PEG-Mannose. Then, cationic lipid and DSPE-PEG-Mannose are respectively dissolved in chloroform-methanol (2:1), and mixed in a certain ratio. The mixture liquid obtained by mixing the both is placed into a round flask and vortically dried with a steady nitrogen stream, so as to form a uniform film, and then placed in a vacuum oven to dry in vacuo overnight. Double distilled water solution containing tumor polyvalent antigen or PBS buffer solution is added on the next day, to place at 4° C. to hydrate for 12 hours. After ultrasonic bath for 10 minutes, the resulting mixture is extruded through polycarbonate film twice, and placed at 4° C. for standby.

Specific operations of preparing dendritic cells loaded with tumor antigen are as follows.

(1) Suspending a human peripheral blood mononuclear cells in basal medium, and then inoculating the human peripheral blood mononuclear cells into cell culture plates for attachment culture for 1-2 hours at 37° C. in incubator.

(2) Washing the non-attached cells away and adding DC cell culture medium into the attachment cells, to culture at 37° C., with 5% of CO₂, under saturated humidity for carrying out the induction of DC cell. On the third day, DC cell culture medium is supplemented to DC cell culture plate in half amount. On the fifth day, a suitable amount of liposome-encapsulated antigen is added to DC cells, cultured for 8-24 hours. On the sixth day, DC maturation promoting medium is added, and continued to culture for 24-48 hours, then DC cells loaded with tumor antigen is obtained.

The present invention uses nano liposome having targeting effect to load tumor antigens to prepare dendritic cell. Compared with the existing technique for preparing DC cell, the technique for preparing DC of the present invention not only uses a new nanoliposome to load polyvalent tumor antigen, so as to overcome problems such as the poor immunogenicity and weak antigenicity of tumor antigen, single immune target, antigen in DC cell existing for a short time; moreover, dendritic cell actively targets to take tumor antigen, enhancing the efficiency of uptake and enrichment of DC to tumor antigen, regulating and controlling the transport and release of the antigen in the DC cell, prolonging the delay-releasing of tumor antigen, promoting the maturation and activation of DC and the ability of antigen-presenting, and enhancing antigen-presenting mediated by MHC-I molecules. In addition, DC preparation method of the present invention is simple and available, and is ease of operation and promotion in the clinical application, in which the immune target is diverse, the antigenicity is strong and the stability is good.

In Example 4, the applicant uses polypeptides with different properties, electroneutrality and electronegativity, indicating that such liposomal carrier can load polypeptides with different properties.

### BRIEF DESCRIPTION OF THE DRAWINGS

FIG. 1 is a structural schematic view of nanovaccine which uses mannose-modified cationic liposomes as the carrier.

FIG. 2 is a view illustrating the preparation of mannose-modified cationic liposome and a characterization diagram thereof.

FIG. 3 is an efficiency diagram of dendritic cell efficiently taking electroneutral polypeptide antigen loaded by a cationic liposome.

FIG. 4 is an efficiency diagram of dendritic cell efficiently taking electronegative polypeptide antigen loaded by a cationic liposome.

FIG. 5 is a diagram indicating the influence of the percentage of DSPE-PEG-Mannose on the efficiency of dendritic cells loaded with tumor antigens.

### DETAILED EMBODIMENT OF THE INVENTION

Embodiments of the present invention will be described in detail below. The example of the embodiment is shown in the figures, wherein the same or similar reference number indicates the same or similar element or elements having the same or similar function throughout. The following embodiments described with reference to the figures are exemplary and are intended to illustrate the invention, but should not be construed as limiting the present invention. Embodiments which are not described with the specific technique or conditions are carried out in accordance with the technique or conditions described in the literature in the art or in accordance with the product instructions. All reagents or equipments, of which the manufacturers are not indicated, are conventional products that can be commercially available

### EXAMPLE 1

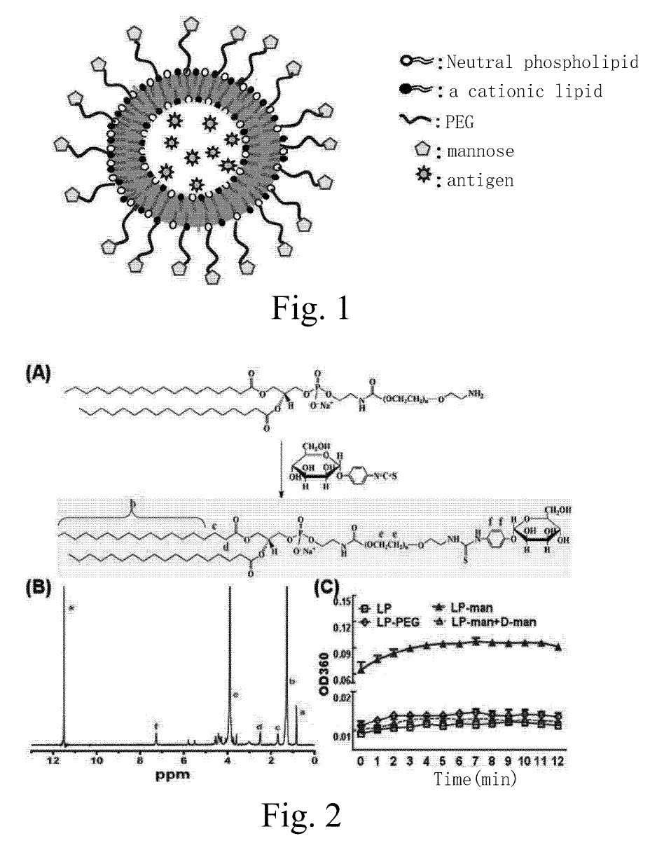

A isothiocyano-α-D-mannopyranose (i.e., α-D-Mannopyranosylphenyl isothiocyanate, Sigma-Aldrich) is reacted with NH₂-PEG-DSPE under basic conditions at room temperature for 24 hours, so that the isothiocyano of mannose and the amino group of PEG are reacted to form a thiourea bond, resulting mannosylated PEG-DSPE (DSPE-PEG-Man, see FIG. 2A). NMR spectrum shows that a peak (f peak) at 7.25 ppm is the benzene ring of the isothiocyano mannopyranose, indicating the mannose is labeled successfully to DSPE-PEG (see FIG. 2B).

The DOTAP and the above DSPE-PEG-Man are dissolved respectively in a mixed solvent of chloroform and methanol of a volume ratio of 2:1 (DOTAP: DSPE-PEG-Man=9:1, mol/mol), and the resulting mixture liquid after mixing the both is placed in a round flask. The mixture liquid is vortically dried with a steady nitrogen stream, so as to form a uniform film, and after drying in vacuo overnight, a PBS buffer solution of antigen protein HBsAg (100 ug antigen/1 umol liposomes/ml, calculated by the feed ratio) is added to place to hydrate at 4° C. on the next day, then a cationic liposome which encapsulates HBsAg is obtained by sonicating the bath for 10 minutes. Concanavalin A (Con A) aggregation tests indicated that a mannose-modified cationic liposome (LP-man) is co-incubated with Con A, and the particle size of the liposome may increase dramatically. On the contrary, an unmodified liposomes (LP) is co-incubated with Con A, and its particle size did not change. This result further confirms that the surface of the liposome is modified with mannose residues (see FIG. 2C).

Human peripheral blood mononuclear cells are suspended in basal medium (10⁶ cells/ml), and then inoculated into cell culture plates for attachment culture for 1-2 hours at 37° C. in incubator; after non-attached cells are removed, a serum-free cell culture medium containing GM-CSF (25-500 ng/m1) and IL-4 (5-100 ng/m1) is added into the attached cells to culture at 37° C., with 5% of CO₂, under saturated humidity for carrying out the induction of DC cells; on the third day, DC cell culture medium is supplemented to DC cell culture plate in half amount; on the fifth day the above-described cationic liposome encapsulating HBsAg (5 ug antigen/50 nmol liposome/ml) is cultured for 8-24 hours, to obtain DC cells loaded with tumor antigen.

### EXAMPLE 2

4-iosthiocyanophenyl-A-D-mannoside is coupled to DSPE-PEG₂₀₀₀ in covalent manner to obtain mannoside-modified DSPE-PEG₂₀₀₀ (DSPE-PEG-Man). The DSPE-PEG-Man and DOTAP are dissolved respectively in a mixed solvent of chloroform and methanol of a volume ratio of 2:1, the ratio of the molar number of N-2 amido glucosamine modified DSPE-PEG₂₀₀₀ to the total molar number of DOTAP is 1:19, and the resulting mixture liquid after mixing the both is placed in a round flask. The mixture liquid is rotarily evaporated with a steady nitrogen stream, so as to form a uniform film. After drying in vacuo overnight, the human hepatocarcinoma cell HepG2 is suspended in PBS buffer solution. After repeatedly freezing (−80° C.) and thawing (70° C.) to disrupt the cells, tumor cell lysate containing tumor antigen is obtained from the supernatant by centrifuging. A certain amount of the tumor cell lysate (0.25 g antigen protein/mol liposome) is added into a phospholipid membrane and to place to hydrate at 4° C., and then the bath is sonicated for 10 minutes, followed by extrusion of a polycarbonate film twice to obtain a cation liposome encapsulating tumor antigens. Mononuclear cells are sampled and separated from human peripheral blood and the DC cell culture medium containing GM-CSF (100 ng/ml) and IL-4 (25 ng/ml) is added, and then placed in an incubator at 37° C., with 5% of CO₂ to culture. 5 days later, the cation liposome encapsulating tumor tissue antigens is added to culture for 8-24 hours, and then the dendritic cells loaded with tumor tissue antigens can be obtained.

### EXAMPLE 3

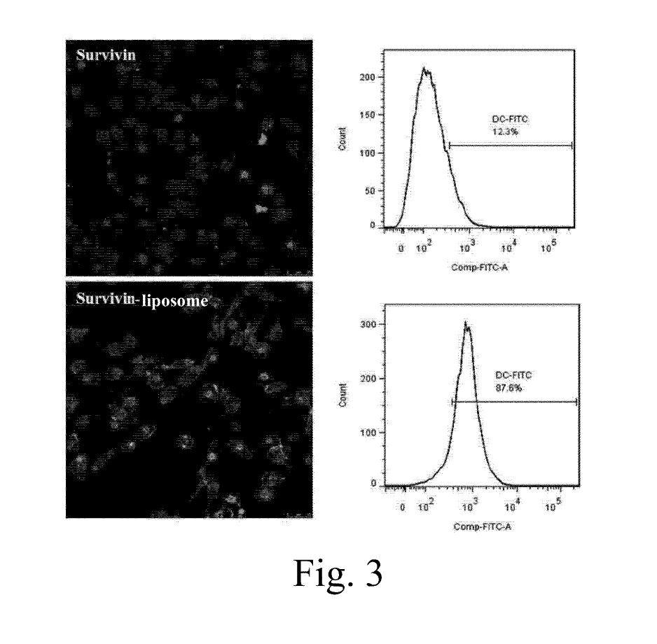

One end of the DSPE-PEG is modified with succinimide (DSPE-PEG-NHS), and then is condensed with 4-aminophenyl-D-mannoside via the amino group and succinimide to form a ester, so as to obtain a mannosyl PEG (DSPE-PEG-Man). The DOTAP and DSPE-PEG-Man are dissolved respectively in a mixed solvent of chloroform and methanol of a volume ratio of 2:1. Then the resulting mixture liquid obtained by mixing the DOTAP and the DSPE-PEG-Man in a molar ratio of 9:1 is placed in a round flask. The mixture liquid is rotarily evaporated with a steady nitrogen stream, so as to form a uniform film, and after drying in vacuo overnight, a PBS buffer solution (0.1 mg protein antigen/mol liposome) containing an electroneutral survivin polypeptide labeled with FITC (survivin-FITC, the sequence of survivin see SEQ ID NO: 1, PI=8.1, MW=3610.19) is added on the next day. And placing to hydrate at 4° C., then a survivin-FITC cationic liposome is obtained by sonicating the bath for 10 minutes followed by extrusion of a polycarbonate film twice. Mononuclear cells are sampled and separated from human peripheral blood and the DC serum-free medium is added, and then the cytokine GM-CSF (100 ng/ml) and IL-4 (25 ng/ml) are added into the medium, and then the medium is placed in an incubator at 37° C., with 5% of CO₂ to culture. The dendritic cells are collected after 5 days; then a nanoliposome encapsulating an electroneutral polypeptide antigen is added and adjusted the concentration to 2.5 μg polypeptide/ml to culture for another 2 hours, and a dendritic cell efficiently loaded with antigen can be obtained as shown in FIG. 3. As can be seen from the figure, according to confocal and flow results, it indicates that the uptake efficiency of DC to the cationic liposome which encapsulates electroneutral polypeptide antigen survivin-FITC is better than that of DC to free electroneutral polypeptide antigen.

### EXAMPLE 4

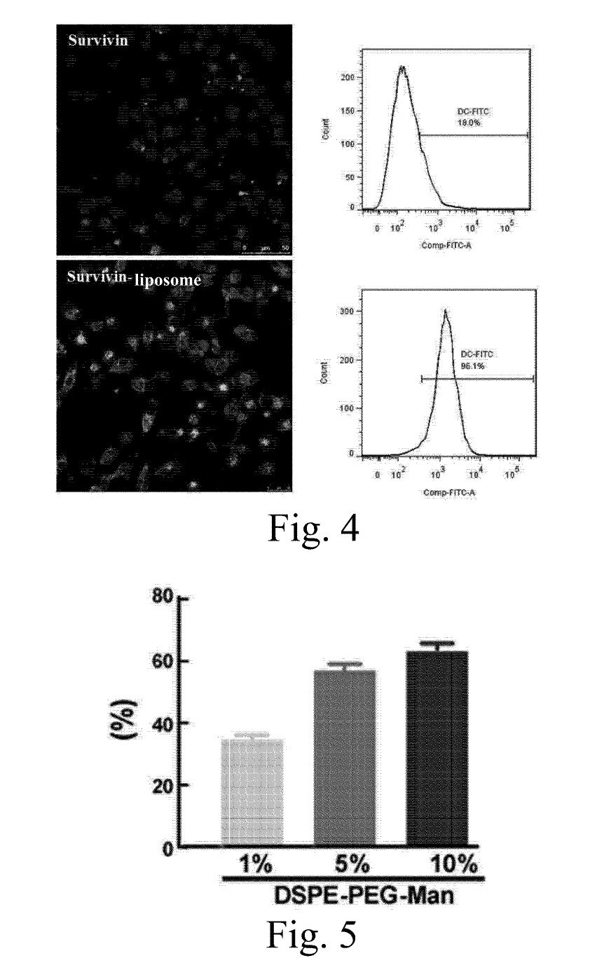

4-iosthiocyanophenyl-A-D-mannoside is coupled to DSPE-PEG₂₀₀₀ in covalent manner to obtain mannoside-modified DSPE-PEG₂₀₀₀ (DSPE-PEG-Man). The DOTAP and DSPE-PEG-Man are dissolved respectively in a mixed solvent of chloroform and methanol of a volume ratio of 2:1. Then the resulting mixture liquid obtained by mixing the DOTAP and the DSPE-PEG-Man in a molar ratio of 19:1 is placed in a round flask. The mixture liquid is rotarily evaporated with a steady nitrogen stream, so as to form a uniform film, and after drying in vacuo overnight, a PBS buffer solution (0.1 mg protein antigen/mol liposome) containing an electronegative polypeptide antigen survivin-FITC, (the sequence of survivin see SEQ ID NO: 1, PI=3.84, MW=3593.96) is added on the next day. And placing to hydrate at 4° C., then a cationic liposome which encapsulates electronegative polypeptide antigen survivin-FITC is obtained by sonicating the bath for 10 minutes followed by extrusion of a polycarbonate film twice. Mononuclear cells are sampled and separated from human peripheral blood and the DC serum-free medium is added, and then the cytokine GM-CSF (100 ng/ml) and IL-4 (25 ng/ml) are added into the medium, and then the medium is placed in an incubator at 37° C., with 5% of CO₂ to culture. The dendritic cells are collected after 5 days; then a nanoliposome encapsulating an electronegative polypeptide antigen is added and to adjust the concentration to 2.5 μg polypeptide/ml to culture for another 2 hours, and then a dendritic cell efficiently loaded with antigen can be obtained, as shown in FIG. 4. As can be seen from FIG. 4, according to confocal and flow results, it indicates that the uptake efficiency of DC to the nanoliposome which encapsulates electronegative polypeptide antigen is better than that of DC to free electronegative polypeptide antigen.

### EXAMPLE 5

4-iosthiocyanophenyl-A-D-mannoside is coupled to DSPE-PEG₂₀₀₀ in covalent manner to obtain mannoside-modified DSPE-PEG₂₀₀₀ (DSPE-PEG-Man). The resulting mixture liquid obtained by mixing the cationic lipid DOTAP and the DSPE-PEG-Man in molar ratios of 9:1, 19:1 and 99:1 is placed in a round flask, so that the molar percentage of DSPE-PEG-Man in the mixture is 1%, 5% and 10%. The mixture is rotarily evaporated with a steady nitrogen stream, so as to form a uniform film, and after drying in vacuo overnight, a PBS buffer solution (0.1 mg protein antigen/mol liposome) containing antigen proteins OVA-FITC is added on the next day. And placing to hydrate at 4° C., then a cationic liposome which encapsulates antigen proteins ovalbumin (OVA-FITC) labeled with fluorescein is obtained by sonicating the bath for 10 minutes followed by extrusion of a polycarbonate film twice. Mononuclear cells are sampled and separated from human peripheral blood and the DC serum-free medium is added, and then the cytokine GM-CSF and IL-4 are added into the medium, and then the medium is placed in an incubator at 37° C., with 5% of CO₂ to culture. The dendritic cells are collected after 5 days; then nanoliposome encapsulating an antigen protein OVA-FITC is added and to adjust the concentration to 2.5 OVA/ml to culture for another 2 hours. Result of Flow cytometry is shown in FIG. 5, and it can be seen from FIG. 5 that as the percentage of DSPE-PEG-Mannose increasing (from 1%, 5% to 10%), the effect of liposome on the efficiency of dendritic cells loading with antigen is more and more significant

Although the above description has been illustrated and described embodiments of the present invention, it is understood that the above-described embodiments are illustrative and are not to be construed as limiting the present invention. One of ordinary skill in the art may make modifications, substitutions and variations to the above-described embodiments within the scope of the present invention, without departing from the principles and spirit of the invention.

1. A preparation method of dendritic cells loaded with antigens efficiently, which is characterized in that: the steps thereof are
adding serum-free cell culture medium containing GM-CSF and IL-4 into mononuclear cells, and placing in an incubator of 37° C., with 5% of CO2 for culture;
after 5 days, adding cationic liposomes that encapsulate target antigen to culture for 8-24 hours, and then dendritic cells loaded with the target antigen can be obtained.

2. The preparation method of dendritic cells loaded with antigens efficiently of claim 1, characterized in that: the cationic liposome is a mannose-modified cationic liposome complex.

3. The preparation method of dendritic cells loaded with antigens efficiently of claim 1, characterized in that: the cationic liposome is obtained by coupling a mannose or a mannoside to a polyethylene glycol derivatized phospholipid to obtain a mannose-modified polyethylene glycol derivatized phospholipid; dissolving a cationic lipid and the mannose-modified polyethylene glycol derivatized phospholipid into a mixed solvent of chloroform and methanol respectively, after mixing to obtain a mixture liquid; rotarily evaporating the mixture liquid with a steady nitrogen stream or an inert gas stream so as to form a uniform film; adding a PBS buffer solution containing tumor antigen after vacuum drying and placing at 4° C. for sonicating to hydrate; obtaining the cationic liposome after extruding through film; wherein the loading amount of tumor antigen is 1-500 g antigen/mol liposome.

4. The preparation method of dendritic cells loaded with antigens efficiently of claim 3, characterized in that: the molar number ratio range of the cationic lipid to the mannose-modified polyethylene glycol derivatized phospholipid is 1:1 to 1:10.

5. The preparation method of dendritic cells loaded with antigens efficiently of claim 3 characterized in that: the cationic lipid is any one of didecyldimethylammonium bromide, dioleoyltrimethylammoniumpropane, dioleoylpropyltrimethylammonium chloride, 3-(N-(N′, N′-dimethylaminoethane) carbamoyl) cholesterol and dioleyl ether phosphatidylcholine.

6. The preparation method of dendritic cells loaded with antigens efficiently of claim 1, characterized in that: the target antigen is one or more tumor antigen protein or polypeptide having different epitopes.

7. The preparation method of dendritic cells loaded with antigens efficiently of claim 6, characterized in that: the antigen may be selected from the group consisting of tumor cell lysate, autologous or allogeneic tumor antigen protein, polypeptide or protein product of genetic engineering and synthetic antigen polypeptide.

8. The preparation method of dendritic cells loaded with antigens efficiently of claim 6, characterized in that: the antigen is HBsAg antigen, tumor tissue antigens, electroneutral polypeptide antigen, electronegative polypeptide antigen survivin or OVA antigen protein.

9. The preparation method of dendritic cells loaded with antigens efficiently of claim 1, characterized in that: the DC cells include peripheral blood mononuclear cell induced DC cells, hematopoietic stem cell and umbilical cord blood stem cell induced DC cells.

10. The preparation method of dendritic cells loaded with antigens efficiently of claim 1, characterized in that: human peripheral blood mononuclear cells are suspended in basal medium, and then inoculated into cell culture plates for attachment culture for 1-2 hours at 37° C. in incubator; after non-attached cells are removed, a serum-free cell culture medium containing GM-CSF and IL-4 is added into the attached cells to culture at 37° C., with 5% of CO2, under saturated humidity for carrying out the induction of DC cells; on the third day, DC cell culture medium is supplemented to DC cell culture plate in half amount; on the fifth day, liposome-encapsulated antigen is added to DC cells, cultured for 8-24 hours, wherein the adjustment dose of the antigen is 1-50 ug/ml, and DC cells loaded with tumor antigens are obtained; preferably, the serum-free cell culture medium contains 25-500 ng/ml of GM-CSF and 5-100 ng/m1 of IL-4.
